(19) United States
(12) Patent Application Publication
(10) Pub. No.: US 2026/0137453 A1
(43) Pub. Date: May 21, 2026

(54) METHOD FOR SELECTING A ROD OF A PEDICLE SCREW SYSTEM FOR SPINAL FUSION

(71) Applicant: MEDACTA INTERNATIONAL SA, Castel San Pietro (CH)

(72) Inventors: Francesco SICCARDI, Castel San Pietro (CH); Meinrad FIECHTER, Castel San Pietro (CH); Denny SINDONI, Castel San Pietro (CH); Gianluca GARGANTINI, Castel San Pietro (CH); Giuliano LAMBERTO, Castel San Pietro (CH)

(21) Appl. No.: 19/118,259

(22) PCT Filed: Jul. 19, 2023
(86) PCT No.: PCT/IB2023/057362

§ 371 Date: Apr. 3, 2025

(30) Foreign Application Priority Data
Oct. 6, 2022 (IT) 102022000020547

(51) Int. Cl. A61B 34/10; A61B 17/70

(52) U.S. Cl. CPC A61B 34/10; A61B 17/7032; A61B 2034/104; A61B 2034/105; A61B 2034/108

(57) ABSTRACT

A method for selecting a rod of a pedicle screw system for spinal fusion is performed by determining the position of a group of screws in a predefined reference system based on the spine of a patient and creating an initial patient rod model connecting the group of screws. Furthermore, it is possible to apply predetermined correction factors to the initial patient rod model and then calculating a final patient rod model based on said correction factors. Finally, the final patient rod model is compared with a database of a plurality of predefined pre-bent rod models and then one predefined pre-bent rod model is selected based on a best fit between the final patient rod model and the plurality of predefined pre-bent rod model.

The present invention relates to a method for selecting a rod of a pedicle screw system for spinal fusion.

Spinal fusion is a surgery treatment to permanently connect two or more vertebrae of a spine, thus eliminating motion between them for stabilizing a vertebral segment of a patient.

Rods are usually used to connect pedicle screws which are inserted into vertebral bodies, to strengthen the spine of a patient.

Pedicle screws are surgical instruments which allow to reinforce spinal fusion in cases of injury of the spine of a patient and they comprise a tulip and a screw.

Curvature abnormality of the spine (scoliosis or kyphosis), spinal vertebrae injury, protrusion of the cushioning disk between vertebrae (slipped disk, herniated nucleus pulposus) and weak or unstable spine caused by diseases are some cases in which spinal fusion is usually applied.

The pedicle screw is a particular type of bone screw designed for implantation into a vertebral pedicle that provides means for grasping a spinal segment at any level of a spinal column (cervical, thoracic and lumbosacral).

A rod is a metal cylinder implant used in spinal surgery to stabilize a vertebral segment, by connecting pedicle screws inserted into adjacent vertebral bodies in order to prevent motion and allow fusion to occur across the disc space.

Usually, rods are bended inside the operating room during surgery, depending on the screw position and the correction required for each patient.

The rod bending process requires a lot of assumption since the rod has to go into the tulip of the pedicle screws, but it is also used as point of contact in order to correct the spine.

In addition to that, bending the rod in the correct way is a complex procedure, especially during a surgical operation, that has to be carried out considering different parameters like the optimal curvature to apply to the rod to achieve a desired correction.

It would therefore be desirable that the patient could receive a rod with an optimal curvature, while reducing the surgical operating time. A patient specific rod or a rod template that can be used as a guide could therefore provide a lot of value to the patient and to the surgeon.

Furthermore, it may happen that due to the properties of the rod itself and/or of the physical condition of the patient, the rod is not able to correctly maintain the desired bended shape.

In this context, for example, a spine which is too stiff may cause an unwanted deformation of the rod once the same is applied that reduces its effectiveness.

There is therefore the need to have a patient specific rod which can be accurately designed, selected and monitored, in particular to verify its conformity to a desired shape based on a tridimensional model of the spine, thus overcoming the problems of the prior art.

These and other objects are fully achieved by virtue of a method for selecting a rod of a pedicle screw system for spinal fusion having the characteristics defined in independent claim 1, and by a system for selecting a rod of a pedicle screw system for spinal fusion having the characteristics defined in claim 15.

Preferred embodiments of the invention are specified in the dependent claims, whose subject-matter is to be understood as forming integral or integrating part of the present description.

Further characteristic and advantages of the present invention will become apparent from the following description, provided merely by way of non-limiting example, with reference to the attached drawings, in which:

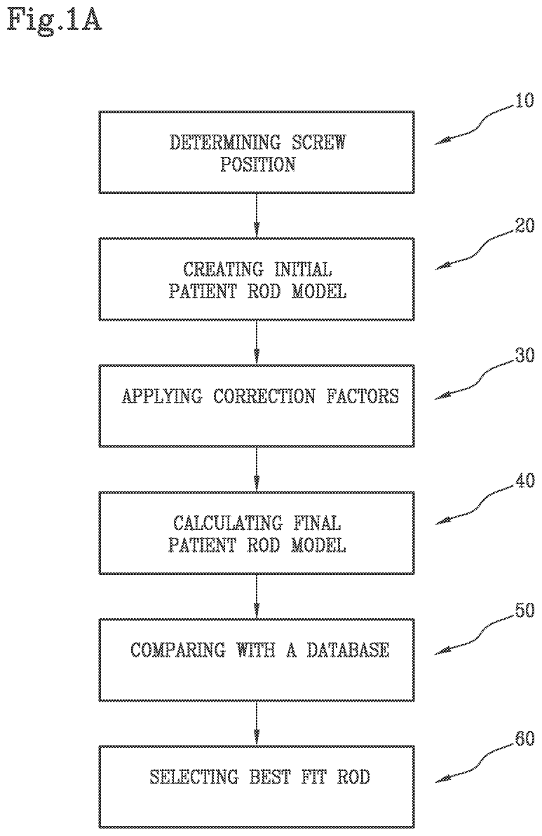

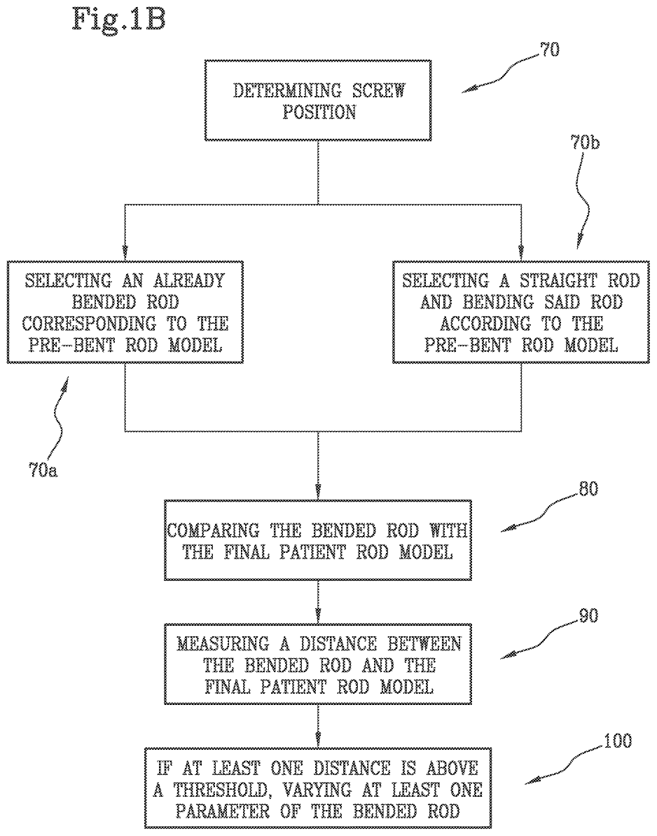

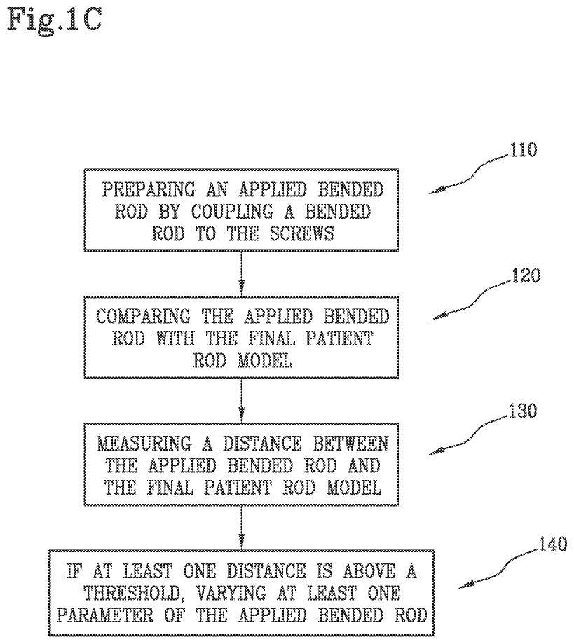

FIGS. 1A-1C show respective block diagram depicting steps of a according to the present invention;

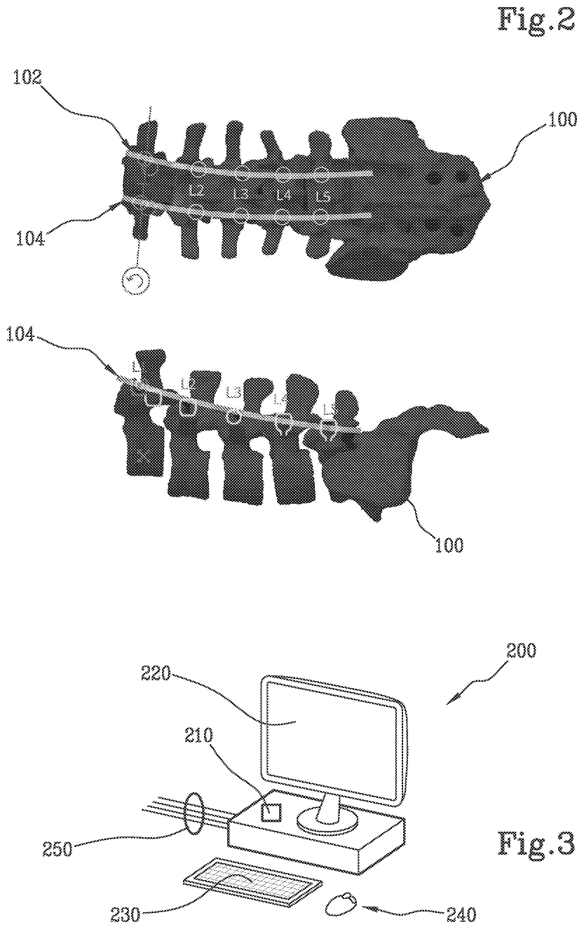

FIG. 2 shows a possible implementing situation for the method of the present invention.

FIG. 3 shows a system for selecting a rod of a pedicle screw system for spinal fusion.

Briefly, in the method for selecting a rod of a pedicle screw system for spinal fusion according to the present invention an optical or electronical tracking technology is used to define the position of screws, specifically pedicle screws, to be implanted into a patient.

In particular, the screws are provided in at least one group composed of all the screws positioned along the spine in a same/corresponding position in each vertebra of interest for a spinal fusion procedure to be carried out.

In other words, the screws are divided into different groups and each group comprises all the screws that in use are aligned or substantially aligned along the spine.

For example, the screws may be all applied along a central axis of each vertebra and thus define just a single group.

Alternatively, the screws may be position along a left and right side of each vertebra thus defining a first group (e.g. the screws positioned on the left side) and a second group (e.g. the screws positioned on the right side).

Then, an initial patient rod model (its shape) is calculated based on the position of a specific group of pedicle screws as well as additional correction factors can be applied based on either pre-operative planning or users experience.

Said initial patient rod model defines the shape that the rod to be coupled to said group need to have in order to efficiently correct the defect that the spinal fusion procedure needs to address.

The correction factors are applied either based on parameters identified during a sagittal balance simulation or based on surgeon assessment and the need for under or over correction of the rod, depending on multiple factors, among others the flexibility of the curve or spine or the correction manoeuvre to perform.

The implementation of the correction factors allows to obtain a final patient rod model that correspond to the ideal shape that the rod has to assume to reach optimal performances once applied.

In a further step, a database is searched, this database comprising a plurality of predefined pre-bent rod models, each having respective curvatures, shapes, materials, lengths different from each other, and then a predefined pre-bent rod model is selected based on a best fit between the final patient rod model and said plurality of predefined pre-bent rod models.

Furthermore, the position where the rod must be cut at the cranial and caudal end is calculated.

Finally, physical rods which correspond to the selected predefined pre-bent rod models are prepared, and then cut according to the plan, in order to be subsequently implanted into the patient.

In general, the preparation of the physical rod may be performed by taking an already bent rod that follows the shape of the selected pre-bent rod model or by selecting a straight rod which is then bended according to the information provided by the pre-bent rod model.

In this context, the pre-bent rod model may comprise or depict specific instructions indicating the bending procedure to be performed on a straight rod to produce the desired shape (e.g., the position of the bending, its direction and its degree).

More in detail, FIG. 1 shows a block diagram of the step of a method for selecting a rod of a pedicle screw system for spinal fusion according to the present invention as above summarized.

In a first step 10, the position of at least one group of screws is determined in a predefined reference system, with an electronical or optical tracking system (a camera or any analogous optical sensor), based on the spine of a patient.

Said optical system may be an external optical system or an optical system positioned and sequentially mounted on each screw to determine its position over the other screws of the same group.

In a second step 20, an initial patient rod model connecting a respective group of screws (which are substantially aligned along a straight line) is created.

With the expression “substantially aligned along a straight line” it is meant that the screws to be connected lie along a line.

As will be discussed more in detail in the following, the whole procedure may be applied to more than just one rod, as could be seen in FIG. 2 which shows a picture of a tridimensional portion of a spine 100 of a patient (top and lateral view) where the references L1, . . . , L5 indicate couple of opposite screws belonging to two distinct and different groups. Lines 102, 104 indicate the initial patient rod model for two different rods which are to be coupled to respective groups of screws.

In a further step 30, correction factors based on a pre-operative planning and/or based on preferences and technique as above detailed are applied to the initial patient rod model.

Then, in step 40, a final patient rod model is calculated, based on the correction factors of step 30.

In a further step 50, the final patient rod model is compared with a database of a plurality of predefined pre-bent rod models and then a predefined pre-bent rod model is selected based on a best fit between the final patient rod model and said plurality of predefined pre-bent rod model. Advantageously, in a further final step 60, the best fit rod is selected from the database, by calculating differences between the selected predefined pre-bent rod models and the final patient rod models and by choosing the rod having the lower difference, and the cranial/caudal location to cut the rods are indicated.

The rod can be from example 600 mm long with a 200 mm lordotic curvature and a 400 mm long kyphotic curvature. However, the patient could need a 375 mm long rod with a 150 mm lordotic curvature and a 225 mm kyphotic curvature. Based on this information the method of the present invention determines to cut 175 mm on top and 50 mm on the bottom.

Further to the above, the method preferably comprises also a step of setting up 70 a bended rod corresponding to the pre-bent rod model.

Said setting up may be performed by preparing a bended rod by selecting 70b a rod according to the pre-bent rod model and bending it so as to replicate the shape of the final patient rod model or alternatively simply by selecting 70a an already bended rod.

It is to say that the starting rod may be a straight rod or an already pre-bended rod which may need (or not) just some minor adjustment to optimally conform with the shape of the final patient rod model.

In other words, the pre-bent rod model may define the rod that requires the less effort/bending to obtain the desired shape which is equivalent to the final patient rod model or provides the instruction to process a straight rod so as to obtain the desired final shape.

Once the pre-bent rod model is defined, it is then possible to select a rod and, only if necessary, bend it so as to replicate the shape of the final patient rod model in a particularly fast and easy way.

Ideally, if a perfect matching between the pre-bent rod model and the final patient rod model is found it is even possible to simply select the corresponding already-bent rod without the need for further operations before coupling it to the corresponding screws.

To guarantee an optimal matching between the final patient rod model and the bended rod it is possible to perform a checking procedure aimed at verifying that the bended rod maintains correctly its deformation and thus maintains the desired shape.

The above checking procedure is performed in particular by comparing 80 the bended rod and the final patient rod model at specific and predefined measurement point and measuring 90 the distances between each measurement point of the bended rod and the corresponding measurement points of the final patient rod model.

Said points may be a continuous set of points so as to perform a checking procedures that verify the entire shape of the rod, or they can correspond to a specific set of reference points of interest, for example the points wherein the rod is to be coupled to the screws (i.e. the position of the tulips), so as to allow for a faster a simpler checking procedure.

In general, if at least one distance is above a predetermined threshold, it is to say if the shape of the rod is too different from the one of the final patient rod model, a step of varying 100 at least one parameter of the bended rod is performed.

Said parameters comprise in particular a shape of at least a portion of the bended rod and consequently the modification of the parameters may be executed by bending at least one portion of the bended rod until the at least one distance, which was too high to be acceptable, is brought below the predetermined threshold.

Alternatively, if it is not possible to produce a satisfactory bended rod even by subsequent bending/correction steps locally applied to specific portion of the bended rod which are found to be too far away from the desired shape, it is also possible to select a different rod and subsequently prepare a new bended rod more suitable to correctly replicate the shape of the final patient rod model.

In this scenario, it would also be possible to repeat the checking procedure also on the new bended rod obtained by selecting and/or bending the different rod.

Specifically, the different rod may be a rod presenting different physical features (such as length, section, materials, starting shape) from the rod originally used.

Advantageously, the claimed method may be implemented to specifically perform the application of a rod in a spinal fusion procedure or in an analogous procedure aimed at improving/stabilizing the spine of a patient. Said rod may be a bended rod prepared and checked following the above-described procedural steps or an already bended rod.

In fact, as discussed above, the pre-bent rod model may correspond to a set of instruction indicating how to bend a straight rod to obtain a certain shape or may simply depict the shape of an already bended rod which is, for example, present in a storage unit and that can be selected and used without the need to perform further preparation steps like any bending and/or checking procedure.

In this context, the method further comprises the step of preparing 110 an applied bended rod by coupling said bended rod to all the screws of the same group.

Operatively, the rod is positioned in contact with the screws pertaining to the same group (i.e. the screws aligned in the same position along the spine) and then coupled and blocked to their tulip.

However, the spine may be too stiff for the applied bended rod causing its deformation.

To guarantee that the rod maintains the correct shape (and that it optimally addresses the problem that required the spinal fusion procedure to be performed) it is possible to perform a further checking procedure analogous and corresponding to the one performed for the bended rod prior to its application on the screws.

In particular, said procedure may be performed by comparing 120 the applied bended rod and the final patient rod at predefined measurement points and measuring 130 the distances between each measurement point of the applied bended rod and the corresponding measurement points of the final patient rod model.

Also in this situation, the comparison between the applied bended rod and the final patient rod may be performed on a continuous set of measurement points that follows the entire length of the rod or just by identifying a discrete set of points.

Preferably, the measurement points correspond to the position of the tulip of all the screws that are connected/coupled to the rod.

In other words, the proposed method may be performed by identifying the position of the screws and calculating a specific rod model according to their position, then, a rod corresponding to the calculated model is selected/prepared and applied, finally it is possible to re-measure the position of the screws (specifically their tulip) to verify if their actual position after the introduction of the bended rod is still the same measured at the beginning of the process when the rod was not present yet.

Again, if at least one distance is above a predetermined threshold, it is to say if the shape of the applied bended rod (and thus the position of the screws) is too different from the one of the final patient rod model, a step of varying 140 at least one parameter of the applied bended rod may be performed.

In this context the variation of the parameters of the applied bended rod may be performed by bending at least one portion of the applied bended rod until said at least one distance is below the predetermined threshold and with said rod still being coupled to the screws.

Otherwise, it is possible to decouple the applied bended rod and either apply to it a further bending or substitute it entirely.

For example, the decoupling of the applied bended rod may be performed by discarding it and selecting a different rod (according to the pre-bent rod model) that is then bended, according to the final patient rod model, and then applied to the corresponding group of screws.

Advantageously, the procedure described above allows to guarantee in any moment that the selected rod (which is then bended and applied to the screws) actually has and maintains the desired shape.

The above-described method may be performed to determine/apply a single rod when such single rod is sufficient to provide the desired effect on the spine of the patient.

Otherwise, it is also possible to perform the above procedures with the aim of providing more than one rod, in particular a couple of rods comprising a first rod to be coupled to a left side of the spine and a second rod to be coupled to a right side of the spine.

In particular, the method may be performed to determine in a predefined reference system based on the spine of the patient the position of a first group, comprising screws positioned on a left side of the spine, and the position of a second group, comprising screws positioned on a right side of the spine.

In a further step, both a left initial patient rod model and a right initial patient rod model are created.

Said models are created so as to connect the first and the second group of screws respectively.

Again, it is possible to apply predetermined correction factors to the left and right initial patient rod models in order to compensate for example potential sagittal balance issue pertaining to the spine of the patient.

Based on the applied correction factor it is possible to calculate a left final and a right final patient rod model.

Finally, by comparing the final left and the final right patient rod models with the above-mentioned database it is possible to select corresponding predefined pre-bent rod models based on a best fit between each of the left final and right final patient rod model and the plurality of predefined pre-bent rod model.

Once the determination of the optimal shape for the rods is made, it is possible to prepare and apply them to the respective group of screws in a sequential way.

It is to say that a second rod (e.g. the rod to be coupled to the right side of the spine) is applied only once a first rod (e.g. the rod to be coupled to the left side of the spine) has been applied and it has been verified that the first rod has maintained its shape.

In particular, the method may be performed preparing a left bended rod by selecting a rod according to the left pre-bent rod model and bending it according to the left final patient rod model and repeating the same procedure in a corresponding way for the right bended rod.

Then both rods may be analyzed through the checking procedure described above in order to verify their ability to maintain the desired shape (i.e. the shape of the corresponding final patient rod model).

Then it is possible to apply the left bended rod by coupling said left bended rod to all the screws of the first group.

Again, the left bended rod may be checked after being coupled to the screws so as to verify that it is not deformed in an unwanted way by any potential force applied on it by the spine itself.

This step is specifically performed by measuring distances between each point of the applied left bended rod and corresponding points of the left final patient rod model.

If all of said distances are below a predetermined threshold, then the rod is behaving in the desired way and as such it is possible to proceed with applying also the right bended rod by coupling it to all the screws of the second group (which is then itself checked in the same manner).

Otherwise, the parameters of the left bended rod are varied (for example using the procedures described above) until its shape corresponds to the one of the left final patient rod model.

After also the right bended rod is applied it is possible to perform on it the same exact checking procedure performed on the left bended rod to verify its ability to maintain the desired shape even under the influence of potential force applied on it by the spine itself.

Alternatively, the two rods may be applied at the same time, being coupled to the respective group of screws and only after both of them are fixed in position the checking procedure is performed.

In general, it is possible to perform the checking procedure sequentially on each rod after the same has been coupled to the respective screws or in one single step performed once both rods have been applied.

The process according to the invention can be in particular performed by a system of the type illustrated in FIG. 2 comprising a workstation 200 of known type having a processing subsystem 210, a display device 220, a keyboard 230, a pointing device (mouse) 240, and a device for connecting to a local area network (network bus) 250.

In FIG. 2 the workstation 200 has been illustrated as a computer, but it may also be any kind of personal device with computing features, like a tablet, a laptop or a mobile phone.

Alternatively, the processing system may be of a distributed type (not illustrated) having a processing subsystem and local or remote peripheral input/output devices.

The workstation 200 or distributed system is arranged to process processing groups or modules and computational programs that are stored on a disk or are accessible over a network and that are suitable for displaying the described process to display results on the device 220.

1. Method for selecting a rod of a pedicle screw system for spinal fusion comprising:
determining a position of at least one group of screws in a predefined reference system based on a the spine of a patient;
creating at least one initial patient rod model connecting a respective group of screws;
applying predetermined correction factors to the initial patient rod model;
calculating a final patient rod model based on said correction factors; and
comparing the final patient rod model with a database of a plurality of predefined pre-bent rod models and then selecting one predefined pre-bent rod model based on a best fit between the final patient rod model and said plurality of predefined pre-bent rod models.

2. The method according to claim 1, further comprising determining differences between the selected predefined pre-bent rod model and the final patient rod model and indicating a cranial and/or a caudal location to cut the rod.

3. The method according to claim 1, comprising setting up a bended rod corresponding to the pre-bent rod model.

4. The method according to claim 3, wherein setting up a bended rod is performed by selecting an already bended rod.

5. The method according to claim 3, wherein setting up a bended rod is performed by selecting a straight rod and bending said straight rod according to the pre-bent rod model so as to replicate the final patient rod model.

6. The method according to claim 3, comprising:
comparing the bended rod and the final patient rod model at predefined measurement points;
measuring distances between each measurement point of the bended rod and the measurement points of the final patient rod model; and
if at least one distance is above a predetermined threshold, varying at least one parameter of the bended rod.

7. The method according to claim 6, wherein varying at least one parameter of the bended rod is performed by
bending at least one portion of the bended rod until said at least one distance is below the predetermined threshold; and/or
selecting a different rod and bending said different rod to replicate a shape of the final patient rod model.

8. The method according to claim 3 comprising preparing an applied bended rod by coupling said bended rod to all the screws of the same group.

9. The method according to claim 8, comprising:
comparing the applied bended rod and the final patient rod model at predefined measurement point;
measuring distances between each measurement point of the applied bended rod and measurement points of the final patient rod model;
if at least one distance is above a predetermined threshold, varying at least one parameter of the applied bended rod.

10. The method according to claim 9, wherein varying at least one parameter of the applied bended rod is performed by:
bending at least one portion of the applied bended rod until said at least one distance is below the predetermined threshold; and/or
decoupling the applied bended rod.

11. The method according to claim 10, wherein decoupling comprises:
discarding the applied bended rod;
selecting a different rod according to the pre-bent rod model;
bending said different rod according to the final patient rod model; and
coupling the different rod to all the screws of the same group.

12. The method according to claim 1, comprising:
determining in a predefined reference system based on the spine of a patient the position a first group of screws comprising screws coupled to a left side of the spine and the position of a second group of screws comprising screws coupled to a right side of the spine;
creating a left initial patient rod model and a right initial patient rod model connecting the first and the second group of screws respectively;
applying predetermined correction factors to said left and right initial patient rod models;
calculating a left final patient rod model and a right final patient rod model based on said correction factors; and
comparing the left and right final patient rod models with a database of a plurality of predefined pre-bent rod models and then selecting corresponding predefined pre-bent rod models based on a best fit between each of the left and right final patient rod model and said plurality of predefined pre-bent rod model.

13. The method according to claim 12, comprising:
preparing a left bended rod by selecting a rod according to the corresponding pre-bent rod model and bending said rod to replicate shape of the left final patient rod model;
preparing a right bended rod selecting a rod according to the corresponding pre-bent rod model and bending said rod to replicate the shape of the right final patient rod model;
applying the left bended rod by coupling said left bended rod to all the screws of the first group; and
applying the right bended rod by coupling said right bended rod to all the screws of the second group, otherwise varying at least one parameter of the left applied bended rod.

14. The method according to claim 13, comprising:
measuring distances between predefined measurement points of the left applied bended rod and corresponding measurement point of the left final patient rod model; and
applying the right bended rod only if all of said distances are below a predetermined threshold.

15. A system for selecting a rod of a pedicle screw system for spinal fusion comprising a workstation having a processing subsystem arranged to:
determine a position of at least one group of screws in a predefined reference system based on a spine of a patient;
create at least one initial patient rod model connecting a respective group of screws;
apply predetermined correction factors to the initial patient rod model;
calculate a final patient rod model based on said correction factors; and
compare the final patient rod model with a database of a plurality of predefined pre-bent rod models and then selecting one predefined pre-bent rod model based on a best fit between the final patient rod model and said plurality of predefined pre-bent rod model.
